(19) United States
(12) Patent Application Publication
(10) Pub. No.: US 2002/0087206 A1
(43) Pub. Date: Jul. 4, 2002

(54) Implantable intracranial photo applicator for long term fractionated photodynamic and radiation therapy in the brain and method of using the same

(76) Inventors: Henry Hirschberg, Oslo (NO); Bruce J. Tromberg, Irvine, CA (US); Steen Madsen, Hendesson, VA (US); Brian K. Pikul, (US)

Correspondence Address: Daniel L. Dawes, MYERS, DAWES & ANDRAS LLP, 5252 Kenilworth Dr., Huntington Beach, CA, 92649, US

(21) Appl. No.: 09/750,832

(22) Filed: Dec. 28, 2000

(51) Int. Cl. A61N 5/067

(52) U.S. Cl. 607/89

(57) ABSTRACT

An inflatable balloon is disposed into a body cavity and inflated to expand into the body cavity. A subcutaneously implanted, resealable catheter is coupled to the inflatable balloon. The resealable catheter provides repetitive access for an optical fiber disposed through a first lumen to illuminate the inner surface and for a radiation source disposed on the distal tip of a wire to provide repetitive long term, fractionated photodynamic and radiation therapy to tissues adjacent to the inner surface. A light diffusing fluid is disposed in the inflatable balloon. The catheter has a self-healing membrane coupled to and closing its proximal end. An insert is coupled to the proximal end and to the first lumen in the subcutaneous, resealable catheter. The insert is funnel shaped, but not necessarily concentric, narrowing down to where the insert is coupled to the lumen to ease in disposition of the insert into the patient and to facilitate introduction of the optical fiber therethrough without damage to the optical fiber. The insert can be disposed into or placed on top and supported only by the bony cranium of the patient and is supported by the cranium so that forces applied to the insert are prevented from being transmitted to underlying brain tissue.

### BACKGROUND OF THE INVENTION

1. Field of the Invention

The invention is a fully implantable indwelling balloon catheter light applicator for photodynamic therapy (PDT).

2. Description of the Prior Art

Conventional techniques of post-operative treatment of residual tumor following only gross removal of tumor include sequential or simultaneous administration of radiation, chemotherapy, and/or heat. Currently available intraoperative therapeutic procedure utilize the cavity formerly occupied by the bulk of the tumor for placement of an inflatable device for subsequent tumor therapy, whether combined (radiation and/or chemotherapy and/or hyperthermia together) or single modality (one of the above alone), or whether simultaneous or sequential in application. See U.S. Pat. No. 6,083,148 (2000) and U.S. Pat. No. 6,022,308 (2000).

Notwithstanding treatment of tumors with radiation, chemotherapy, and/or heat, the poor prognosis for patients with malignant brain neoplasm has led to a search for better treatment modalities. Although gliomas are considered to be disseminated tumors in the brain, 80-90% recur within 2 cm of the site of the previous tumor resection. Improved local control would therefore be of clear benefit.

Intracavity therapy offers the possibility of applying various treatment modalities (brachy, photodynamic, thermal therapies) aimed at the nests of tumor cells left in the resection border while minimizing damage to normal tissue. This in turn requires that the shape of the resection cavity be stabilized, i.e. not folded in on itself, and geometrically simple to insure uniform irradiation over a relatively long time period. Furthermore it would be very advantageous if the stabilized cavity is accessible percutaneously to allow repeated treatment sessions over long time intervals. What is needed is an apparatus and methodology by which these goals are achieved.

### BRIEF SUMMARY OF THE INVENTION

A liquid filled balloon stabilizes a resection cavity in the brain which ensures a constant and simple geometric shape throughout the treatment period. It also provides for an even light distribution to the walls of the resection cavity in the brain. The device allows for repeated percutant introduction of an optical fiber into the balloon center for transmission of laser or other light energy into the brain or other parts of the body. This device allows for repeated and long term PDT that can be delivered in repeated fractions over a long time period. Additionally it maintains sterility since there are no explanted elements penetrating the skin when the device is not in use.

More specifically the invention is defined as an apparatus for placement in a body cavity having an inner surface in a patient. the apparatus comprising an implantable, inflatable balloon for disposition into the body cavity. When inflated the balloon expands into the body cavity to prevent the inner surface of the body cavity from folding in on itself and to thus allow substantially all of the inner surface to be exposed to at least one point within the balloon. A subcutaneous, implantable catheter is coupled to the inflatable balloon for percutant disposition into the patient to access the body cavity. The catheter is arranged and configured to provide repetitive access to the body cavity over an extended period of time. The catheter has a first lumen to allow an optical fiber to be disposed through the first lumen into the inflatable balloon to illuminate the inner surface of the body cavity to provide repetitive photodynamic therapy to tissues adjacent to the inner surface of the body cavity.

The apparatus further comprises a light diffusing fluid disposed in the inflatable balloon and an optical fiber coupled into the fluid in the balloon. The optical fiber has a distal end and a light diffuser disposed on its distal end. The subcutaneous catheter has a proximal end and a self-sealing membrane, which is composed of a silicone elastomer as is found in many subcutaneous implanted devices, coupled to and closing its proximal end. The subcutaneous catheter has an insert coupled to its proximal end. The insert in turn has a distal end coupled to the first lumen in the subcutaneous catheter. The first lumen has a distal end and a transparent plug disposed in its distal end which seals the first lumen. The catheter has a second lumen defined therethrough which is used to inflate the balloon. A valve seals the second lumen to prevent deflation of the balloon. The insert is funnel shaped, narrowing down to where the insert is coupled to the lumen to ease the disposition of the insert into the patient and to facilitate introduction of the optical fiber therethrough without damage to the optical fiber. The insert snugly press fits into the lumen, and disposed into and supported only by a cranium of the patient and is supported by the cranium so that forces applied to the insert are prevented from being transmitted to underlying brain tissue.

In one embodiment the apparatus is entirely subcutaneously implanted. In another embodiment the apparatus is entirely subcutaneously implanted in a breast.

The apparatus further comprises an ambulatory laser and control circuit for repetitive, fractionated photodynamic treatment. The laser and all or part of its control circuit may also be external to and nonambulatory with the apparatus.

In another embodiment the apparatus further comprises a detector for recording dosage levels and the history of dosages applied to the patient by the ambulatory laser and control circuit.

The apparatus further comprises a radiation source disposable in the catheter for repetitive, fractionated radiation treatment in combination with fractionated photodynamic treatment through the catheter. The radiation source is a wire disposable into the catheter with a distal tip having a radioactive material disposed thereon. In one embodiment a subdermally implanted remote optical coupler and a permanently implanted optical fiber is provided communicating between the optical coupler and the balloon. In one version the subdermally implanted remote optical coupler is entirely subdermally implanted. In another version the subdermally implanted remote optical coupler further comprises a transdermal optical connector.

The invention is also characterized as a method of photodynamically treating a tumor resection characterized by a body cavity having an inner surface in a patient comprising the steps of selectively disposing and retaining a photosensitizing drug in cancerous tissue within the inner surface of the body cavity and adjacent thereto; disposing an inflatable balloon into the body cavity coupled to a subcutaneous catheter; inflating the inflatable balloon in the body cavity by means of a lumen in the wall of the subcutaneous catheter to prevent the inner surface of the body cavity from folding in on itself and to thus allow substantially all of the inner surface to be exposed to at least one point within the balloon; disposing an optical fiber through the subcutaneous catheter to position a distal end of the optical fiber within the inflatable balloon; and repetitively delivering a fractionated dosage of light through the optical fiber to effectively photodynamically treat the tumor resection.

The method further comprises the step of removing the optical fiber from the subcutaneous catheter. The method continues by repeating the disposition of the optical fiber into the subcutaneous catheter and the delivering a dosage of light through the optical fiber to effectively photodynamically treat the tumor resection during treatments repeated over an extended period of time. The method is distinguished in that the extended period of time comprises at least one month or more than one year.

The step of inflating the inflatable balloon in the body cavity by means of a lumen in the wall of the subcutaneous catheter inflates the balloon with a light diffusing fluid. The optical fiber is positioned or positionable in the balloon over an extended period of time during which the a fractionated dosage of light is repetitively delivered. Again the extended period of time comprises at least one month to more than one year.

In addition an ambulatory laser and control circuit can be provided to the patient, which laser and circuit are coupled to the optical fiber to repetitively deliver a fractionated dosage of light through the optical fiber to effectively photodynamically treat the tumor resection.

The method further comprising disposing a radiation source through the subcutaneous catheter to position a distal end of the radiation source within the inflatable balloon, and repetitively delivering a fractionated dosage of radiation from the radiation source in combination with a repetitively delivered fractionated dosage of light through the optical fiber to effectively photodynamically treat the tumor resection.

In another embodiment the step of disposing the optical fiber through the subcutaneous catheter comprises disposing the optical fiber through an implanted remote access port. In one embodiment the optical fiber is coupled to an optical coupler serving as the remote access port and a permanent implanted optical fiber couples the optical coupler to a light emission point positioned in the balloon. The step of repetitively delivering a fractionated dosage of light through the optical fiber thus comprises the steps of coupling an external optical fiber to the optical coupler and delivering the fractionated dosage of light through the external optical fiber to the optical coupler.

In one of the illustrated embodiments the step of coupling an external optical fiber to the optical coupler and delivering the fractionated dosage of light through the external optical fiber to the optical coupler comprises coupling the external optical fiber with the optical coupler by transdermal disposition of the external optical fiber.

In another embodiment the step of coupling an external optical fiber to the optical coupler and delivering the fractionated dosage of light through the external optical fiber to the optical coupler comprises coupling the external optical fiber with the optical coupler by coupling to an optical connector which extends transdermally from the optical coupler.

While the method has been described for the sake of grammatical fluidity as steps, it is to be expressly understood that the claims are not to be construed as limited in any way by the construction of “means” or “steps” limitations under 35 USC 112, but to be accorded the full scope of the meaning and equivalents of the definition provided by the claims. The invention can be better visualized by turning now to the following drawings wherein like elements are referenced by like numerals.

### BRIEF DESCRIPTION OF THE DRAWINGS

FIG. 1 is a diagrammatic side cross-sectional view of the invention shown implanted into a human brain without the introduction of an optical fiber therein.

FIG. 2 is a diagrammatic side cross-sectional view of the invention shown implanted into a human brain with the introduction of an optical fiber therein.

FIG. 3 is a diagrammatic side cross-sectional view of the invention shown implanted into a human breast for use in long term fractionated, low dose PDT treatment.

FIG. 4 is a diagrammatic side cross-sectional view of the invention wherein radiation and PDT treatments are given in a synergistic combination of long term fractionated, low dose treatments.

FIG. 5 is a diagrammatic side cross-sectional view of the invention wherein a remotely implanted optical coupler is used in combination with a permanently implanted optical fiber to provide long term fractionated, low dose PDT treatments.

FIG. 6 is a diagrammatic side cross-sectional view of the invention wherein a remotely implanted optical coupler and a transdermal optical connector is used in combination with a permanently implanted optical fiber to provide long term fractionated, low dose PDT treatments.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

PDT is a form of local cancer treatment in which cell death is caused by photochemical reactions involving an exogenous photosensitizer. The photosensitizer, which is preferentially retained in malignant tissues, is photoactivated and cell death results from the generation of reactive products—most likely singlet oxygen. Accurate PDT dosimetry requires knowledge of photosensitizer concentration, tissue oxygenation status, and light fluence (measured in Joules/cm²).

Photodynamic therapy (PDT) is comprised of two phases: the selective uptake and retention of a photosensitizing drug by the tumor followed by drug activation by light. Previous measurements have demonstrated that the applicator described here is suitable for use in PDT, a treatment modality that depends, in large part on adequate and uniform light distribution in the surrounding tissue.

An inflatable balloon is disposed into a body cavity and inflated to expand into the body cavity to prevent the inner surface of the body cavity from folding in on itself and to allow substantially all of the inner surface to be exposed to light emitted from within the balloon. A subcutaneously implanted, resealable catheter is coupled to the inflatable balloon. The resealable catheter provides repetitive access for an optical fiber disposed through a first lumen to illuminate the inner surface to provide repetitive photodynamic therapy to tissues adjacent to the inner surface and for a radiation source disposed on the distal tip of a wire to provide repetitive radiation therapy to tissues adjacent to the inner surface. A light diffusing fluid is disposed in the inflatable balloon. The subcutaneous, resealable catheter has a self-healing membrane coupled to and closing its proximal end. An insert is coupled to the proximal end and to the first lumen in the subcutaneous, resealable catheter. A second lumen is used to inflate the balloon. The insert is funnel shaped, but not necessarily concentric, narrowing down to where the insert is coupled to the lumen to ease in disposition of the insert into the patient and to facilitate introduction of the optical fiber therethrough without damage to the optical fiber. The insert can be disposed into or placed on top and supported only by the bony cranium of the patient and is supported by the cranium so that forces applied to the insert are prevented from being transmitted to underlying brain tissue. The invention further includes the method of using the apparatus for long term photodynamic therapy.

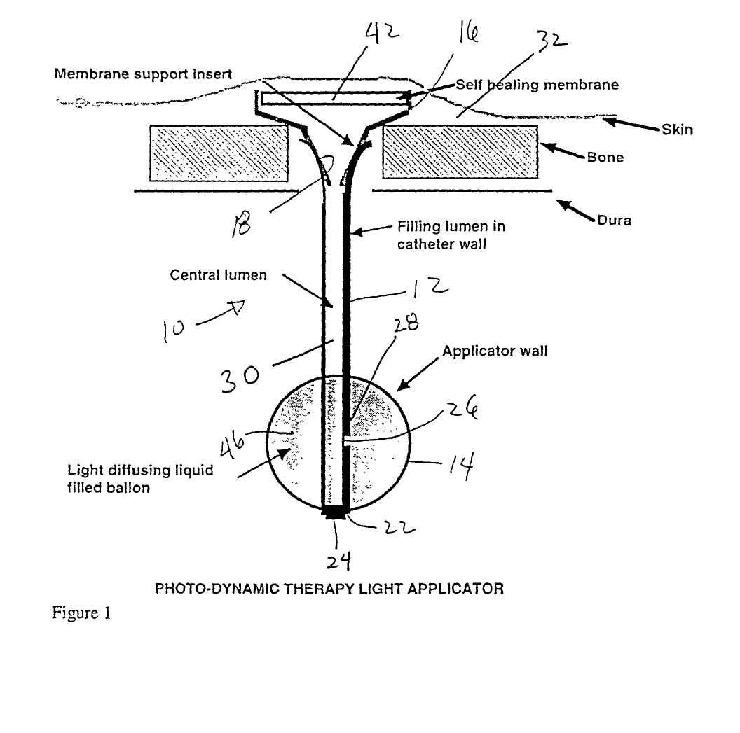

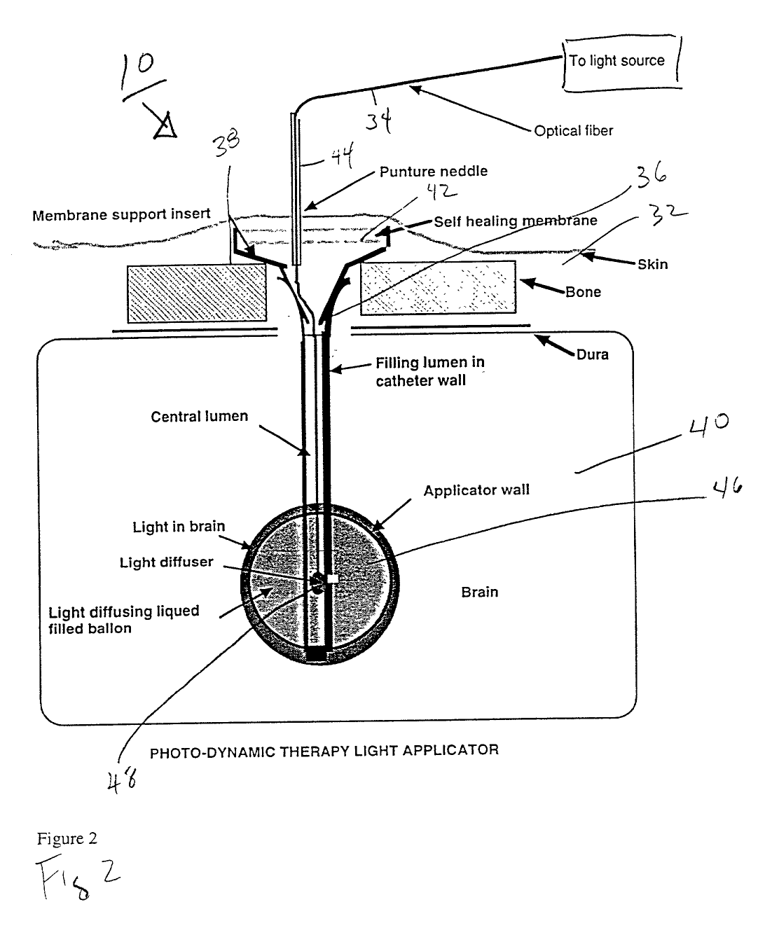

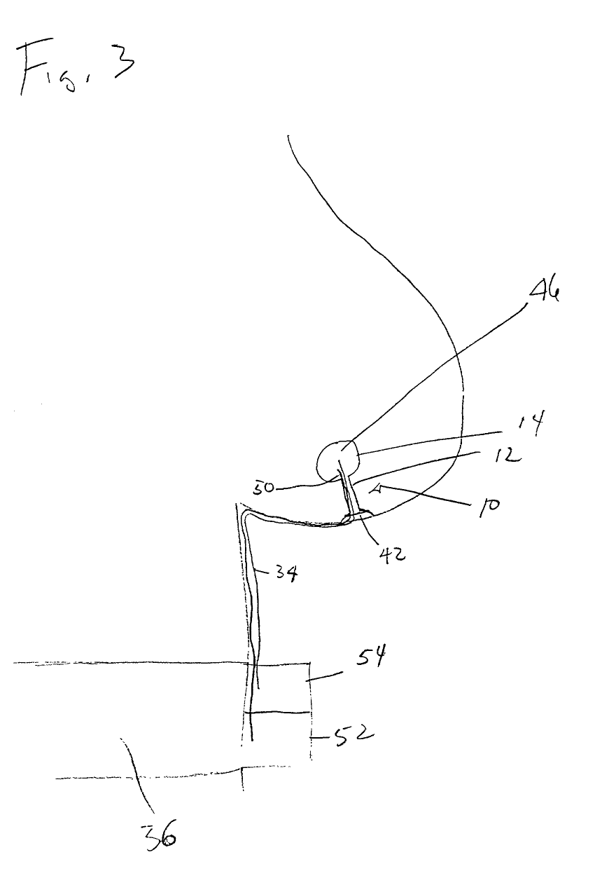

What is disclosed is an indwelling balloon applicator 10 for postoperative intracavity afterloading for photodynamic therapy (PDT) as shown in FIGS. 1 and 2. The light distribution surrounding a balloon catheter applicator 10 was sufficiently uniform to be used in postoperative PDT of malignant brain neoplasms. Following tumor resection, applicator 10 is positioned in the center of the resultant cavity and a balloon 14 is inflated with a scattering solution 46. The liquid-filled balloon 14 stabilizes the resection cavity ensuring a constant and simple geometric shape during treatment. The applicator 10 and catheter 12 are implanted subdermally for use in extended treatment. Although the illustrated embodiment is described as being an intracranial implantation any body implantation is included within the scope of the invention, such as breast implantation following a lumpectomy or any other surgical procedure as diagrammatically depicted in FIG. 3.

Light (wavelength of 630 nm) from an argon-ion pumped dye laser (not shown) is coupled into an optical fiber 34 in FIG. 2 which was inserted into balloon applicator 10. Two different light delivery fibers were investigated: a 15 mm long cylindrical diffuser, and a 1.4 mm spherical diameter diffuser. Balloon 14 was in one embodiment filled to a diameter of 3 cm with either saline or a 0.1% Intralipid™ (Kabivitrum, Inc., Clayton, N.C.) scattering solution.

In a phantom study, balloon 14 was immersed in a 2% Intralipid-filled phantom which simulated the optical scattering characteristics of human brain tissue. Light levels were measured with a 0.8 mm diameter spherical tipped optical detector fiber 34 positioned in contact with the applicator balloon surface. A lock-in detection technique was used to minimize the effect of background noise. Prior to each measurement, light output fluctuations of the laser were monitored and found to be within 3%.

The light intensity (or, more appropriately, the irradiance) was measured as a function of position along the Measurements of light distribution in a phantom model surrounding the balloon catheter 12, show that it may be used to deliver sufficiently uniform light doses during PDT. The light distribution is uniform to within 5% when balloon 14 is filled with a scattering medium. Based on simple assumptions, it is shown that applicator 10 can be used to deliver a sufficient optical dose to brain tissue at a depth of 1 cm in less than 1 hr.

The light intensity (or, more appropriately, the irradiance) was measured as a function of position along the balloon catheter (at 45° intervals from pole-to-pole), type of source fiber (spherical or cylindrical diffuser), type of catheter-filling fluid (saline or 0.1% Interlipid), and position of source fiber in applicator 10 (geometric center or lower pole).

In the case of the saline-filled balloon with the spherical diffuser at the center, the irradiance is uniform to within 5% except at an angle of 0°. The 30% decrease in irradiance at this angle is probably due to the inhomogeneity of the irradiance emanating from the spherical diffuser tip. The presence of the fiber prevents emission of light from the diffuser in the backward direction, hence, the amount of light reaching the detector at 0° will be reduced. In the case of the cylindrical diffuser, the irradiance is approximately 12% higher at 0°. This is probably a distance effect, i.e., the detector-to-source fiber distance is at a minimum at this angle. The addition of a scattering solution to the applicator improves significantly the uniformity of the irradiance. In this case, the irradiance is uniform to within 5% at all measured locations for both spherical and cylindrical diffusers positioned in the center of the applicator.

Although both types of diffusing tip fibers yield equally uniform irradiation patterns in the case of the Intralipid-filled applicator 10, a higher absolute light output level is obtained with the spherical diffuser regardless of filling solution. This is attributed to a superior light coupling efficiency of the spherical diffuser. In all cases, the measured irradiance is 5-10% lower in the case of the Intralipid-filled balloon. This is due to absorption of light by Intralipid.

When the source is placed at the center of the applicator 10, measured irradiances are independent of detector position since the distance between source and detector is always the same. Conversely, when the source is placed at the bottom of the applicator, measured irradiances increase as the detector is moved towards the bottom of the applicator. The observation of irradiances greater than 100% for angles in excess of 90° is due simply to the fact that all signals are normalized to the signal obtained for the spherical source fiber located in the center of the saline-filled balloon (detector fiber at 90ø).

These results are consistent with a distinct change in the uniformity of the light distribution as a function of source fiber position in a balloon applicator 10. If the fiber was positioned past the geometric center of the balloon, the forward hemisphere appeared brighter than the backward hemisphere, and vice versa. This is of clinical relevance since it suggests that it is possible to change the light distribution in the resected cavity simply by changing the position of the source within the applicator 10.

The degree of uniformity of the irradiance is very dependent on the applicator diameter, and on the concentration of Intralipid in applicator 10. Improved uniformity may be achieved with larger diameter balloons 14 and/or higher concentrations of Intralipid 46. It is important to note, however, that absorption of light increases with increasing Intralipid concentration. The balloon diameter and Intralipid concentration used in the phantom study demonstrated sufficient uniformity and acceptably small absorption for diameters of 3 cm and Intralipid concentrations of 0.1%.

Having demonstrated an acceptable uniformity (±5% ) in the light distribution surrounding applicator 10, attempts were made to estimate the time required for delivery of an adequate optical dose during PDT treatment of the brain. Such an estimate requires knowledge of the light propagation characteristics in biological tissue. Light propagation in turbid media, such as biological tissues, has been the subject of intense research in recent years.

For photodynamic therapy to be efficacious a minimum “threshold” optical dose is required. Although the threshold level is dependent on a number of factors 30-50 J/cm² is typical for many photosensitizer/tissue combinations. PDT treatment times can be estimated using the measured light delivery characteristics of the applicator and the known light absorbing and scattering characteristics of the brain. For a 30 mm applicator, a 2 Watt 635 nm laser and a 1 cm brain penetration beyond the balloon surface, treatment times of the order of 40 min are adequate to obtain 50 J/cm². However to reach comparable light levels at a 2 cm brain penetration depth, more then 20 hrs are required.

In a scattering medium, such as biological tissue, the light distribution will be almost isotropic ally distributed at distances sufficiently far from sources and boundaries. The spatial distribution of the optical fluence rate (W/cm²) can then be adequately described by diffusion theory, and the optical dose (fluence) may be determined by solving the diffusion equation for the particular geometry under consideration. In the case of a spherical applicator positioned centrally in a spherical cavity, the optical distribution is given by

Φ=Φ₀e^{-r/δ}/r

where Φ₀ is the fluence rate at the inner surface of the cavity, r the distance from the center of the cavity, and δ the optical penetration depth.

Multiple reflections will build up the fluence rate in the cavity until the radiation transmitted through the cavity wall is equal to the light coupled into the cavity. The observation of increased light fluence rates in hollow geometries due to multiply scattered light is known as the integrating sphere effect. It is important to account for this effect as it reduces treatment times due to the fluence build up. Furthermore, in a true integrating sphere effect, the irradiance is independent of source position in applicator 10. In the presence of an integrating sphere effect, the optical dose (fluence) may be given by:

ψ=[Pₜct/4πaDr(1/a+1/δ)]e^{-(r-a)/δ} (1)

where ψ is the optical dose (J/cm²), Pₜ the total optical power from the applicator, c the velocity of light in tissue, t the treatment time, a the radius of the spherical cavity, and D the diffusion constant. Both the diffusion constant and the optical penetration depth depend on tissue absorption and scattering characteristics.

The calculated treatment times do not account for photodecomposition of the sensitizer during irradiation; a phenomenon known as photobleaching. The effect of photobleaching is to reduce the optical dose due to the fact that light causes destruction of the photosensitizer. Photobleaching is particularly relevant in tissues close to the applicator 10 (i.e., a few mm) where fluences are high, but is of minor importance far from the source. For example, at a depth of 2 mm into brain tissue, the effective dose may be half of that calculated from Equation (1) below, while at a depth of 1 cm, the effective and calculated doses are equivalent. Thus, neglecting the effects of photobleaching, results in an overestimate of optical dose (and subsequent tissue damage) in tissues close to the applicator.

Accurate determination of optical dose requires knowledge of the optical properties (scattering and absorption) of the brain. However, there is significant patient-to-patient variability in optical properties.

For example, a four-fold difference in tumor penetration depth in five patients has been noted in the art. This large patient-to-patient variability suggests that optical properties must be determined for each patient prior to initiation of treatment. Furthermore, the optical properties and, hence the fluence distribution, may change during treatment due to alterations in blood flow and/or to edema. As a result, real-time monitoring of the optical fluence distribution may be necessary in order to achieve sufficient accuracy in the optical dose. This can be accomplished through the insertion of isotropic fiber detectors into the treatment volume. Such measurements have been confined mostly to excised human tissue. Heretofore, due to the invasiveness, and amount of time required for accurate fiber placement and verification, only a few limited in vivo measurements have been attempted. Prior to the present invention real-time monitoring is particularly problematic in organs such as brain, where the presence of additional interstitial probes can damage viable tissue.

Accordingly the use of an indwelling implantable delivery system allowing long treatment times and multiple fractionation of both drug and light delivery provide substantial flexibility in designing and optimizing clinical treatment protocols. All earlier attempts to utilize PDT in the treatment of brain tumors were limited to “one shot” intraoperative applications and have proved disappointing. The ability to give treatment repetitively over a long time period, such as months to years, is a primary achievement of the invention described here. Also it has been shown that low fluence rates are advantageous for PDT. This is difficult to obtain intraoperatively due to normal time constraints. An indwelling light applicator, on the other hand lends itself well to long treatment sessions and low fluence rates.

The applicator 10 of the invention is comprised of two parts: a two lumen silicone catheter 12 with an attached inflatable balloon 14, and a solid (plastic or metal) insert 16 that fits into the central lumen tip 18 and supports a self healing penetrable membrane 20 as shown diagrammatically in side cross-sectional view in FIG. 1. The balloon catheter can be similar to a conventional silicone Foley catheter, but other designs would serve the purpose as well.

The distal end 22 of the central lumen is sealed just bellow the end of the balloon 14 by a transparent silicon plug 24. The end of the balloon filling lumen 26 residing in the catheter wall 28 is also sealed off to prevent leakage and balloon deflation. The length of the catheter 10 is determined during the surgical implantation and must be amenable to in situ determination and modification, this is true of both the central lumen 30 as well as the balloon inflating lumen 26. In other embodiments discussed below, different or more variable lengths are permitted by the use of a remote or distant access site so that lead length may not need to be so closely controlled. The membrane support insert 16 is funnel shaped to allow ease of penetration through the skin 32 and guides the optical fiber 34 shown in FIG. 2 into central lumen 30 in a non traumatic fashion so as not to damage fiber 34. The distal end 36 of the membrane support insert 16 is inserted into the end of the of the central lumen 30 as shown expanding catheter 10 and resulting in a snug fit holding it in place. Insert 16 sits on the cranial bony surface 38 so that no forces are transmitted into brain 40. The entire device is covered by intact skin 32 thus preserving sterility, and greatly reducing the risk of infection. Since the central lumen 30 is sealed at both ends it has no contact with brain 40, csf or other biological tissue or fluids. The central lumen 30 is air filled and not liquid filled, and is further not maintained under pressure. By this means the central lumen 30 is segregated from the interior of balloon 14 and its fluid contents 46, which is inserted into the interior of the balloon during surgery through a separate secondary lumen 26 defined in the wall of catheter 10. Thus, the interior of balloon 14 is kept sterile and isolated from the interior of catheter 10, namely lumen 30, into which optical fibers and other devices are repeatedly inserted and withdrawn from exterior to the body. Therefore, in this sense catheter 10 of the invention is after-loaded with the radiation sources, light sources or other mediation devices with which therapeutic processes are performed.

After implantation and wound healing the top of the device is palpated through skin 32 and skin 32 and membrane 42 are punctured with a puncture needle 44 with mandrill (not shown). The mandrill is removed and fiber 34 is threaded down into the center of the balloon 14 as shown in FIG. 2. The distal end of fiber 34 is provided with a light diffuser 48. Balloon 14 is filled through lumen 30 with a light diffusing liquid 46 to assist in the even illumination of the surrounding tissue. After treatment fiber 34 is withdrawn.

At the termination of the total treatment period the device can be removed under local anesthesia and a small skin incision. Balloon 14 is deflated and the entire device is drawn out. The skin incision is sutured.

The invention is thus characterized by defining a stable cavity to be irradiated. It allows uniform distribution of the radiant energy to the surrounding tissue, and is completely implanted and does not penetrate the skin after implantation. It allows for repeated access to the cavity to be irradiated over relatively long time periods, such as months to years, via simple skin puncture. Since the device is indwelling long treatment times can become practical thus allow low fluence rates which greatly increase the efficacy of PDT. The device has the potential to be modified to allow the direct delivery of the radiation sensitizer or photo sensitizer to the tissue being treated. It can be employed in other sites than the brain, and can be removed in a simple, relatively nontraumatic manner.

As mentioned above, the apparatus of the invention may be modified to include a light detector as part of the implanted device externally connected or communicated to external electronic recording instruments so that either a historical phototherapy record may be created or cumulative dosage recorded.

Further, the therapy may be automated so that photodynamic treatment is repeated automatically by a connected instrumentation and laser source which is ambulatory with the patient. This allows the dosage to be fractionated and repetition rates to be conveniently increased over longer treatment periods, which protocol appears to improve the efficacy of the photodynamic treatment. Thus, it is within the scope of the invention that by such means the photodynamic treatment may be repeated thousands of times over a period of a year or more to effectively inhibit or eliminate the regrowth of cancerous tissue which cannot be practically surgically removed from the resection. FIG. 3 illustrates both an implanted light detector 50 and ambulatory microminiaturized recording instrument 52, and an ambulatory microminiaturized laser and control circuit 54 carried by the patient in a harness or belt 56 to facilitate low dosage, fractionated, repetitive photodynamic treatment of a human breast or any other application site over an extended time period.

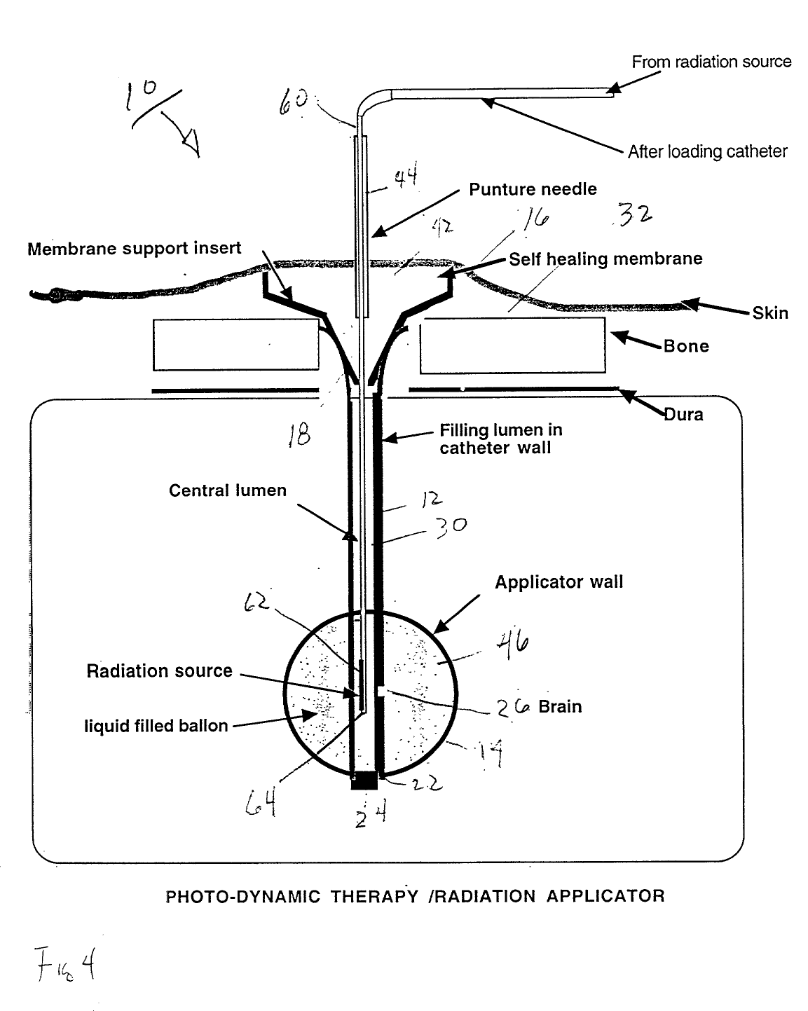

FIG. 4 is a diagrammatic side cross-sectional view of another embodiment in which radiation therapy is combined with PDT. In this embodiment puncture needle 44 is used to deliver a wire 60 through subdermal membrane 42 into lumen 30 of catheter 10. Wire 60 has a conventional radioactive source 62 disposed on its distal tip 64 which is advanced into balloon 14. Typically, source 62 is shielded in a containment or shielding vessel (not shown) and its removed from the containment vessel only under controlled circumstances to expose the patient to a therapeutic dose. After the radiation exposure is completed, wire 60 is withdrawn from catheter 10 for storage in its containment vessel. In a similar manner and as described above, an optical fiber 34 as shown in FIG. 2 may be inserted into lumen 30 of catheter 10 either before or after the radiation treatment to provide a light dose to the tissues. While it is not yet clearly understood, there is a synergistic effect between radiation treatments and PDT treatments, and there appears to be threshold doses of both radiation and light which may be related to each other as well. In addition the cumulative effect of frequent smaller doses of combined radiation and PDT doses appears to have interrelated effects which can materially affect the efficacy of the treatment. The ability to remove the radiation source 62 as shown in FIG. 4 from the site provides significant safety benefits compare to the prior art approach of having an in situ radioactive liquid in balloon 14 and allows for a controlled combined radiation and PDT therapy which exploits the synergistic effects of this combined treatment approach.

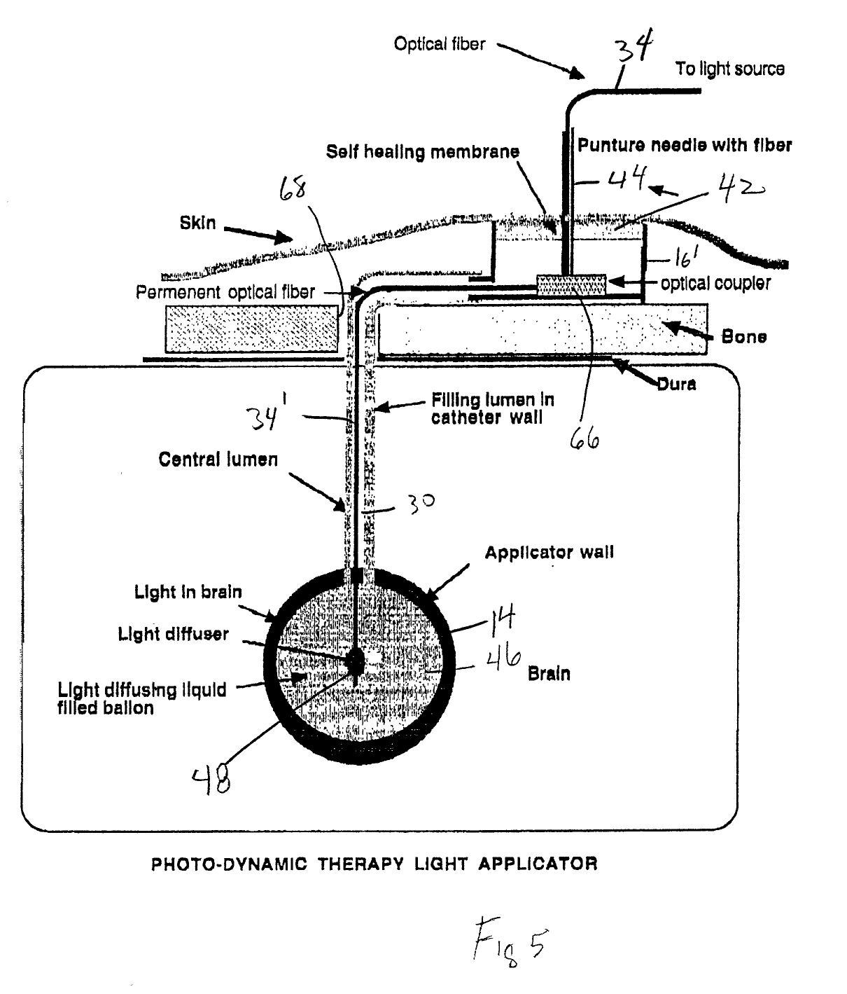

FIG. 5 is a diagrammatic side cross-sectional view of another embodiment in which a completely subdermal, implanted catheter 10 utilizes a remote or distant access to lumen 30. In this embodiment a permanent optical fiber 34′ is disposed in lumen 30 and positioned in balloon 14. Membrane support insert 16 is replaced by a remote housing 16′ which is subdermally implanted. Optical fiber 34′ is permanently coupled to an optical coupler 66. Thus, when the external optical fiber 34 is disposed through membrane 42 by means of needle 44, it is coupled into optical coupler 66. The desire light dose is then transmitted from the external laser or source (not shown) through optical fiber 34, to optical coupler 66, to implanted optical fiber 34′ and thence to light diffuser 48. Remote housing 16′ can thus be situated as some distance on the patient's body away from the cranial access bore 68 since optical fiber 34′ is thin and very flexible. For example, remote housing 16′ can even be implanted subdermally in the chest wall in a manner similar to conventional cardiac pacemakers.

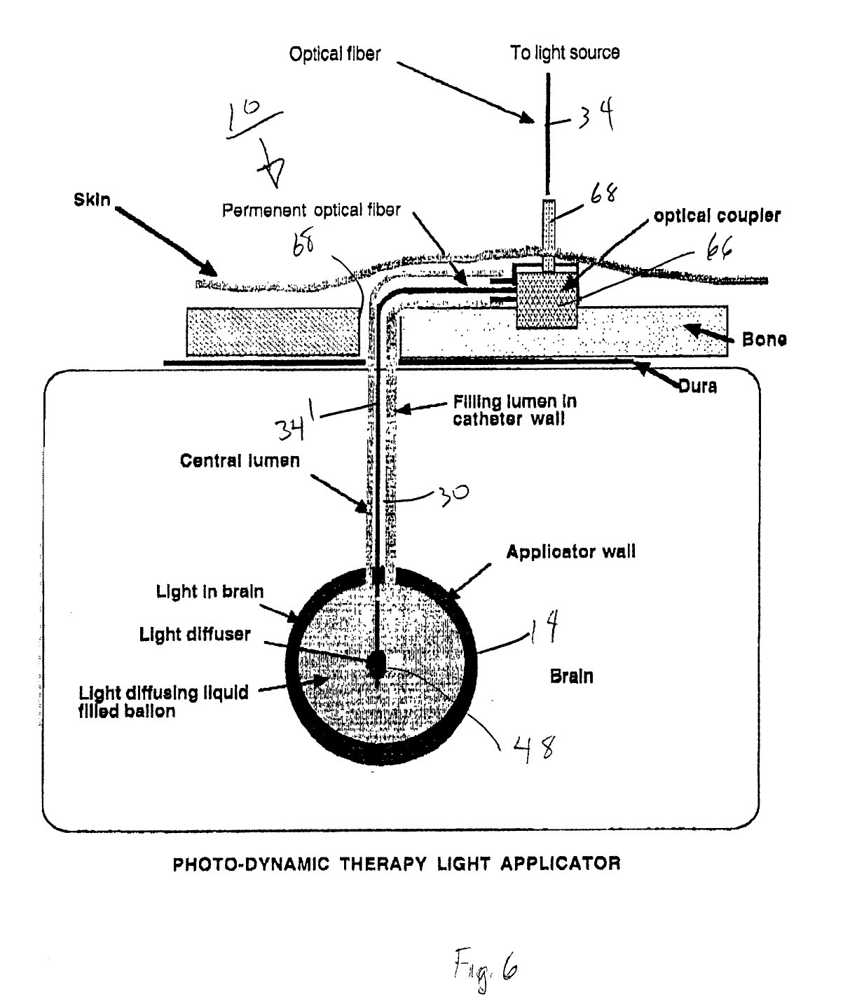

FIG. 6 is a diagrammatic side cross-sectional view of another embodiment in which the embodiment of FIG. 5 is provide with a transdermal optical connector 68. In this embodiment, optical coupler 66 is provided within an input which penetrates the skin to allow connection with fiber 34 outside of the body. The connection may include a physical connection of fiber 34 with transdermal optical connector 68 or may be essentially or completely optical. For example, transdermal optical connector 68 may be an optical window with or without any means of mechanical connection to external fiber 34. In the case where skin is transparent or nearly transparent to the frequency of light used, optical connector 68 and/or optical coupler 66 may actually be implanted just below the skin surface and may not penetrate the skin.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An apparatus for placement in a body cavity having an inner surface in a patient, said apparatus comprising:
an implantable, inflatable balloon for disposition into said body cavity and which when inflated expands into said body cavity to prevent said inner surface of said body cavity from folding in on itself and to thus allow substantially all of said inner surface to be exposed to at least one point within an interior of said balloon; and
a subcutaneous, implantable catheter coupled to said inflatable balloon for percutant disposition into said patient to access said body cavity, said catheter arranged and configured to provide repetitive access to said body cavity over an extended period of time, and having an first lumen to allow an optical fiber to be disposed through said first lumen into said inflatable balloon while being segregated from said interior of said balloon and to illuminate said inner surface to provide repetitive photodynamic therapy to tissues adjacent to said inner surface, and having a second lumen for inflation of said balloon.

2. The apparatus of claim 1 further comprising a light diffusing fluid disposed in said inflatable balloon.

3. The apparatus of claim 1 further comprising said optical fiber.

4. The apparatus of claim 3 where said optical fiber has a distal end and further comprising a light diffuser disposed on said distal end of said optical fiber.

5. The apparatus of claim 1 wherein said subcutaneous catheter comprises a proximal end and a self-healing membrane coupled to and closing said proximal end.

6. The apparatus of claim 1 wherein said subcutaneous catheter comprises a proximal end and comprises an insert coupled to said proximal end, said insert having a distal end coupled to said first lumen in said subcutaneous catheter.

7. The apparatus of claim 1 wherein said first lumen has a distal end and further comprising a transparent plug disposed in said distal end of said first lumen and sealing said first lumen.

8. The apparatus of claim 1 wherein said subcutaneous catheter has a second lumen defined therethrough used to inflate said balloon.

9. The apparatus of claim 8 further comprising a valve for sealing said second lumen to prevent deflation of said balloon.

10. The apparatus of claim 6 wherein said insert is funnel shaped, narrowing down to where said insert is coupled to said lumen to ease in disposition of said insert into said patient and to facilitate introduction of said optical fiber therethrough without damage to said optical fiber.

11. The apparatus of claim 10 where said insert snugly press fits into said lumen.

12. The apparatus of claim 11 where said insert is disposed into and supported only by a cranium of said patient and is supported by said cranium so that forces applied to said insert are prevented from being transmitted to underlying brain tissue.

13. The apparatus of claim 1 wherein said apparatus is entirely subcutaneously implanted.

14. The apparatus of claim 1 wherein said apparatus is entirely subcutaneously implanted in a breast.

15. The apparatus of claim 1 wherein said apparatus further comprises an ambulatory laser and control circuit for repetitive, fractionated photodynamic treatment.

16. The apparatus of claim 15 wherein said apparatus further comprises a detector for recording dosage levels and history applied to said patient by said ambulatory laser and control circuit.

17. The apparatus of claim 1 further comprising a radiation source disposable in said catheter for repetitive, fractionated radiation treatment in combination with fractionated photodynamic treatment through said catheter.

18. The apparatus of claim 17 where said radiation source is a wire disposable into said catheter with a distal tip having a radioactive material disposed thereon.

19. The apparatus of claim 1 further comprising a subdermally implanted remote optical coupler and a permanently implanted optical fiber communicating between said optical coupler and said balloon.

20. The apparatus of claim 19 where said subdermally implanted remote optical coupler is entirely subdermally implanted.

21. The apparatus of claim 19 where said subdermally implanted remote optical coupler further comprises a transdermal optical connector.

22. A method of photodynamically treating a tumor resection characterized by a body cavity having an inner surface in a patient comprising:
selectively disposing and retaining a photosensitizing drug in cancerous tissue within said inner surface of said body cavity and adjacent thereto;
disposing an inflatable balloon into said body cavity coupled to a subcutaneous catheter;
inflating said inflatable balloon in said body cavity by means of a first lumen defined in said subcutaneous catheter to prevent said inner surface of said body cavity from folding in on itself and to thus allow substantially all of said inner surface to be exposed to at least one point within said balloon;
disposing an optical fiber through a second lumen defined in said subcutaneous catheter to position a distal end of said optical fiber within said inflatable balloon; and
repetitively delivering a fractionated dosage of light through said optical fiber to effectively photodynamically treat said tumor resection.

23. The method of claim 22 further comprising removing said optical fiber from said subcutaneous catheter.

24. The method of claim 23 further comprising repeating the disposition of said optical fiber into said subcutaneous catheter and the delivering a dosage of light through said optical fiber to effectively photodynamically treat said tumor resection during treatments repeated over an extended period of time.

25. The method of claim 24 where said extended period of time comprises at least one month.

26. The method of claim 24 where said extended period of time comprises more than one year.

27. The method of claim 22 where inflating said inflatable balloon in said body cavity through said subcutaneous catheter inflates said balloon with a light diffusing fluid.

28. The method of claim 22 where disposing an optical fiber through said subcutaneous catheter repetitively positions said optical fiber therein over an extended period of time during which said a fractionated dosage of light is repetitively delivered.

29. The method of claim 28 where said extended period of time comprises at least one month.

30. The method of claim 28 where said extended period of time comprises more than one year.

31. The method of claim 22 further comprising providing an ambulatory laser and control circuit to said patient coupled to said optical fiber to repetitively deliver a fractionated dosage of light through said optical fiber to effectively photodynamically treat said tumor resection.

32. The method of claim 22 further comprising disposing a radiation source through said subcutaneous catheter to position a distal end of said radiation source within said inflatable balloon, and repetitively delivering a fractionated dosage of radiation from said radiation source in combination with a repetitively delivered fractionated dosage of light through said optical fiber to effectively photodynamically treat said tumor resection.

33. The method of claim 22 where disposing said optical fiber through said subcutaneous catheter comprises disposing said optical fiber through an implanted remote access port.

34. The method of claim 33 where disposing said optical fiber through a remote access port disposes said optical fiber to an optical coupler serving as said remote access port and having a permanent implanted optical fiber coupling said optical coupler to a light emission point positioned in said balloon, and where repetitively delivering a fractionated dosage of light through said optical fiber comprises coupling an external optical fiber to said optical coupler and delivering said fractionated dosage of light through said external optical fiber to said optical coupler.

35. The method of claim 34 where coupling an external optical fiber to said optical coupler and delivering said fractionated dosage of light through said external optical fiber to said optical coupler comprises coupling said external optical fiber with said optical coupler by transdermal disposition of said external optical fiber.

36. The method of claim 34 where coupling an external optical fiber to said optical coupler and delivering said fractionated dosage of light through said external optical fiber to said optical coupler comprises coupling said external optical fiber with said optical coupler by coupling to an optical connector which extends transdermally from said optical coupler.
